(19) United States
(12) Patent Application Publication
(10) Pub. No.: US 2003/0181889 A1
(43) Pub. Date: Sep. 25, 2003

(54) Healing accelerator

(76) Inventor: Adrian Leigh Little, Orlando, FL (US)

Correspondence Address: ADRIAN L. LITTLE, 1216 OLD ENGLAND LOOP, SANFORD, FL, 32771, US

(21) Appl. No.: 10/102,317

(22) Filed: Mar. 21, 2002

(51) Int. Cl. A61K 9/22

(52) U.S. Cl. 604/891.1; 424/424; 602/48

(57) ABSTRACT

The Healing Accelerator is a bio-absorbable device that takes internal wound healing to a new dimension. The Healing Accelerator supplies otherwise nutrient-deprived connective tissues of a animals, i.e. ligaments and tendons, with the necessary growth factors to begin the regeneration and growth process that must occur for healing to successfully take place. The Healing Accelerator cannot only be used for the storing and diffusion of growth factors, but it also can be used to deplore drugs in specific regions of the body. The Healing Accelerator shall take various shapes and forms dependent upon the location of the area of concern in the body, i.e. wound. The Healing Accelerator shall be a permeable membrane that allows the diffusion of growth factors such as VEGE or drugs/medicine into the damaged blood vessels and tissue. By deploying these factors into nutrient-starved regions of the body, the healing process is initiated earlier and the time needed for full recovery is thus lessened. This reduction in healing time will in consequence, reduce costs incurred by the insurance companies and the like.

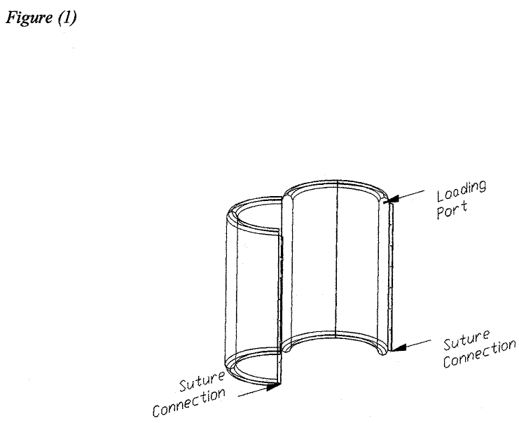

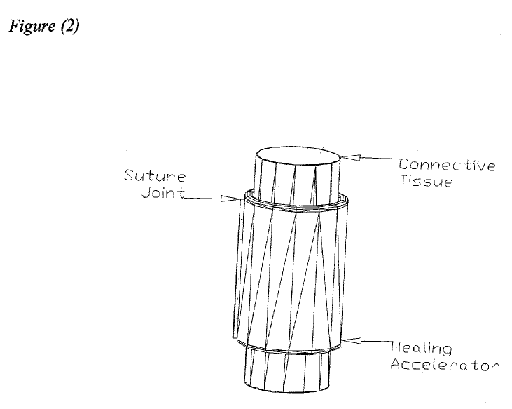

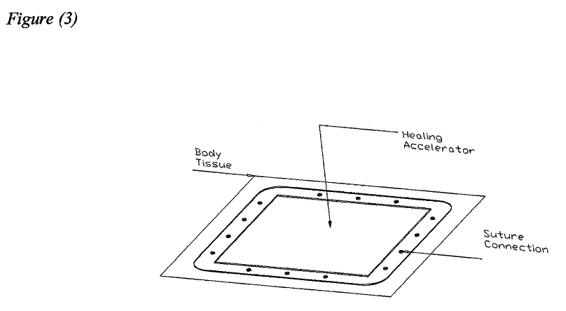

### BACKGROUND OF THE INVENTION

Wound Healing

With the recent boom of exercise/sports and transportation by automobile in our society, connective tissue injuries have increased in the past 10-20 years. In the United States, there are approximately 200,000 people who have ligament tears repaired each year. These costs total over 3.5 billion dollars. Seventy-percent of these people have anterior cruciate ligament tears.

The recovery time from connective tissue injuries are dependent upon the type of injury, patients age, physical condition, and health. Typical recovery time for anterior cruciate ligament tears averages between six to eight weeks. The healing time for Achilles tendon ruptures is an estimated six months.

The process of wound healing contains three important steps beginning with angiogenesis followed by fibroplasias, and culminating with collagen formation/fibrosis.

When connective tissue is ruptured or torn, blood vessels supplying the tissue are ruptured or torn also. The body seeks to begin angiogenesis, which is the process of forming and differentiating blood vessels. When tissue is small, (<1 mm diameter), mass transport to and from the region will occur by diffusion. This diffusion supplies the tissue with vital nutrients and waste removal essential for growth and development of new tissue. The growth and development of the tissue is highly dependent upon the tissue's ability to effectively engage in mass transportation, i.e. diffusion.

The first step of angiogenesis is hemostasis. Hemostasis is initiated through the incurrence of vascular spasms, platelet plugging, blood coagulation and finally the growth of fibrous tissue. Thrombocytes (platelets) carried in the blood assist in the prevention of hemorrhaging. These thrombocytes originate from the blood marrow of the bones and have a life of eight to eleven days in circulating blood. These thrombocytes contain platelet-derived growth factor (PDGF), which causes the growth and multiplication of fibroblasts, vascular endothelial and smooth muscle cells. PDGF also originates from endothelial cells, macrophages, monocytes, and smooth muscle cells. PDGF stimulates angiogenesis and collagen production.

When trauma occurs to the sub-endothelial cells and collagen is exposed, thrombocytes adhere to the trauma site. Platelet and fibrinogen adhesion is initiated by the release of adenosine diphosphate (ADP), serotonin, and thromboxane A2. Fibrinogen adhesion is favored by the vascular permeability of newly reinforced vessels and vascular endothelial growth factor (VEGF). These platelet plugs have receptors that are waiting for the coagulation factors.

Once the platelet plug has formed, coagulation can begin. Coagulation involves the formation of fibrin to reinforce the platelet plug. These coagulants are always present in the blood along with anti-coagulants. These normally dormant coagulants become activated at the formation of a platelet plug.

Next, the initial matrix is replaced by type III collagen and cross-linked by tissue fibronectin. Myofibroblasts contain actin filaments, myosin and smooth muscle cell properties. These myofibroblasts contract the wound and further aid the heating process.

Lastly, Type I collagen replaces the type III collagen to improve the tensile strength of the wounded region. Collagenase removes type III collagen in conjunction with type I collagen's synthesis in response to TGF-β (Transforming Growth Factors-Beta). Type I collagen is a major component of bone, and is the dominant type of collagen in a scar.

Vulpeau stated that the tendon sheath is vital to the healing process of the tendon. This sheath allows the repair to undergo extrinsic healing. Extrinsic healing was reported by Potenza. Potenza was able to prove that the growth of granulation tissue originated from structures located outside the tendon. Bergljung also concluded that the healing nutrients of the tendon were supplied by the paratenon. Heil et al reported that VEGF stimulates the monocyte migration through endothelial monolayers and that this monocyte migration increased with the increase of VEGF concentration.

(Jozsa and Kannus)

Mass Transport

Fick's first and second law shall be used to describe the mass transportation process of the nutrients because of a concentration gradient. Fick's first law in mathematical terms is

$$J = - D \frac{\partial C}{\partial X} \qquad Eq . ( 1 )$$

where:

J: mass flux (g/s m²)

D: diffusion coefficient (m²/s)

C: concentration (g/m³)

X: direction of mass transport (m)

Fick's Second Law adds a time component and states that the rate of change of concentration in a volume, within the diffusional field, is proportional to the rate of change of concentration gradient at that point in the field, as given by:

$${R}_{A} + D {∇}^{2} C = \frac{\partial C}{\partial t} \qquad Eq . ( 2 )$$

where:

t: time

RA: molar rate of production of A per unit volume

Equation (2) can be further expanded and solved for Cartesian, cylindrical, and spherical coordinates.

(Bird, Stewart, and Lightfoot)

### BRIEF SUMMARY OF THE INVENTION

The Healing Accelerator is a bio-absorbable, thermoplastic device that can be implanted into a living animal that has sustained trauma to connective tissues. These connective tissues are typically of low blood supply and therefore lack nutrients required for fast healing. The Healing Accelerator shall contain growth factors such as (VEGF), connective tissue growth factor (CTGF), monocytes, or medicine. The Healing Accelerator shall allow for the time or concentration dependent deployment of its contents. In the case of the growth factor, the amount of damaged cells, and thus the concentration of VEGF will determine the rate of release of the monocytes from the Healing Accelerator and thus the rate of arterogenesis. This accelerated healing shall lead to the patient's ability to undergo early motion treatment sooner than ever before.

The Healing Accelerator shall also be capable of internally deploying medicine to a patient. The Healing Accelerator shall allow for the localizing of vital medicine to a region of trauma. The medicine shall be able to diffuse through the permeable Healing Accelerator housing and make direct contact with the tissue of concern. The intimate contact of the Healing Accelerator shall increase the rate of healing.

### REFERENCES

Bergljung, L (1968). Vascular reaction after tendon suture and tendon transplantation. A steromicroangiographic study on the calcaneal tendon of the rabbit. Scand J Plast Reconstr Surg Suppl. 4, 7-63.

Bird, Stewart, and Lightfoot. Transport Phenomena, 1960.

Jozsa and Kanus, Human Tendons: Anatomy, Physiology, and Pathology, 1997.

Heil, M., Clauss, M, Suzuki, K Buschmann, I. Willuweit, A. Fischer, S. Schaper, W. Vascular endothelial growth factor stimulates monocyte migration through endothelial monolayers via increased integrin expression. European Journal of Cell Biology 79, 850-857.

Potenza A D. Tendon healing within the flexor digital sheath in the dog: An experimental study. J Bone Joint Surg (Am) 44,49-64.

Vulpeau S (1839). Orthopedic Surgery. London.

### BRIEF THE SEVERAL VIEWS OF THE DRAWING

FIG. (1): Illustrates the loading/open position of the cylindrically shaped Healing Accelerator

FIG. (2): Depicts the Healing Accelerator installed around connective tissue.

FIG. (3): Illustrates the planar shaped Healing Accelerator installed.

### DETAILED DESCRIPTION OF INVENTION

The Healing Accelerator shall contain any/all types of chemicals related to the healing and regeneration of body tissue. For example, monocytes suspended in solution such as saline, various growth factors (VEGF, CTGF), or medication. The Healing Accelerator shall be of either cylindrical or plate shape. For instance, monocytes shall be extracted from the patient's blood during the pre-surgical evaluation and loaded into the Healing Accelerator by a syringe. Diffusion shall occur radially from a volume of high concentration (OD) to a volume of low concentration (ID). The concentration of monocytes pre-loaded into the Healing Accelerator shall be predetermined during the pre-surgical analysis. Some factors that will determine the proper concentration of monocytes are, the patients age, physical condition, and health. The deployment of the monocytes shall accelerate the arterogenesis without causing excess collagen formation.

The thermoplastic Healing Accelerator can be processed by blow-molding, calendaring, or extrusion into a flexible hollow cylindrical or plate shape. The connective flaps shall be made of the same bio-absorbable, thermoplastic material as the nutrient housing. Suture indentations shall be made in the connective suture flaps to aid the physician in suturing.

Upon installation, the physician shall suture the flap ends together around/to the connective tissue. During healing, the lacerated blood vessels shall react with the monocytes as previously mentioned. The non-toxic Healing Accelerator housing shall dissolve into the body as the monocyte concentration is reduced.

The Healing Accelerator is a life-changing improvement to wound care. It allows the physician to deploy various nutrients restricting them to a local region of the body bypassing the circulating blood. The Healing Accelerator also allows the physician to apply the nutrients where the patient needs the nutrients the most. The Healing Accelerator is completely bio-absorbable and non-toxic. Not only shall physicians use the Healing Accelerator for humans, but veterinarians shall also v be able to apply the Healing Accelerator to equines, canines, felines, and the like.

1. I claim the concept and use of inserting nutrients i.e. growth factors or medication into the bio-absorbable healing accelerator housing device for use in humans and any animal.
